(19) United States
(12) Patent Application Publication
(10) Pub. No.: US 2025/0186781 A1
(43) Pub. Date: Jun. 12, 2025

(54) ELECTRONIC DEVICE AND METHOD FOR DETERMINING INTENSITY OF LOW-FREQUENCY CURRENT

(71) Applicant: Acer Incorporated, New Taipei City (TW)

(72) Inventors: Yi-Jin Huang, New Taipei City (TW); Yin-Hsong Hsu, New Taipei City (TW); Wei-Hao Chang, New Taipei City (TW); Chien-Hung Li, New Taipei City (TW)

(73) Assignee: Acer Incorporated, New Taipei City (TW)

(21) Appl. No.: 19/055,525

(22) Filed: Feb. 18, 2025

Related U.S. Application Data
(62) Division of application No. 17/243,539, filed on Apr. 28, 2021, now Pat. No. 12,263,345.

(30) Foreign Application Priority Data
Mar. 18, 2021 (TW) 110109782

(51) Int. Cl. A61N 1/36; A61B 5/395; A61B 5/397

(52) U.S. Cl. CPC A61N 1/36171; A61B 5/395; A61B 5/397; A61N 1/36139

(57) ABSTRACT

An electronic device and a method for determining the intensity of a low-frequency current are provided. The method includes: individually applying a corresponding first current to a body part of a user in N consecutive time intervals, wherein the time intervals include an i-th time interval to an (i+N)-th time interval; obtaining electromyography values of the body part in each time interval; determining a second current corresponding to an (i+N+1)-th time interval based on the first current corresponding to each time interval, the body part, personal information of the user, and the electromyography values of each time interval; and applying a second current to the body part of the user in the (i+N+1)-th time interval.

### CROSS-REFERENCE TO RELATED APPLICATION

This application is a divisional application of and claims the priority benefit of U.S. application Ser. No. 17/243,539, filed on Apr. 28, 2021, which claims the priority benefit of Taiwan application serial no. 110109782, filed on Mar. 18, 2021. The entirety of each of the above-mentioned patent applications is hereby incorporated by reference herein and made a part of this specification.

### BACKGROUND

### Technical Field

The disclosure relates to a technique for determining the current intensity, and in particular to a method for determining the intensity of a low-frequency current and an electronic device.

### Description of Related Art

In rehabilitation departments or relevant medical places, electrotherapy of patients through electrotherapy instrument has become a very common medical treatment. In addition, there are also various low-frequency electrotherapy instrument on the market for the user to perform electrotherapy on their own according to requirements after purchase.

In the medical places, most of the electrotherapy instrument are controlled by relevant medical personnel. However, the user must control the current intensity of the low-frequency electrotherapy instrument on their own when using the low-frequency electrotherapy instrument on their own. Generally speaking, the medical effects provided by the low-frequency electrotherapy instrument are not directly related to the current intensity thereof, and an excessive current intensity may sometimes cause harm to the user.

Therefore, for persons skilled in the art, how to design a mechanism for automatically determining the intensity of a low-frequency current is indeed an important topic.

### SUMMARY

The disclosure provides a method for determining the intensity of a low-frequency current and an electronic device, which can be used to solve the above technical issue.

The disclosure provides a method for determining the intensity of a low-frequency current, which is applicable to an electronic device and includes the following steps. A corresponding first current is individually applied to a body part of a user through an electrode patch in N consecutive time intervals. The time intervals include an i-th time interval to an (i+N)-th time interval, where i and N are positive integers. Multiple electromyography values of the body part in each time interval are obtained through an electromyography value measurement circuit. A second current corresponding to an (i+N+1)-th time interval is determined based on the first current corresponding to each time interval, the body part, at least one personal information of the user, and the electromyography values of each time interval. The second current is applied to the body part of the user through the electrode patch in the (i+N+1)-th time interval.

The disclosure provides an electronic device, which includes an electromyography value measurement circuit, a storage circuit, an electrode patch, and a processor. The storage circuit stores a code. The processor is coupled to the electromyography value measurement circuit, the storage circuit, and the electrode patch, and accesses the code to execute the following steps. A corresponding first current is individually applied to a body part of a user through an electrode patch in N consecutive time intervals. The time intervals include an i-th time interval to an (i+N)-th time interval, where i and N are positive integers. Multiple electromyography values of the body part in each time interval are obtained through an electromyography value measurement circuit. A second current corresponding to an (i+N+1)-th time interval is determined based on the first current corresponding to each time interval, the body part, at least one personal information of the user, and the electromyography values of each time interval. The second current is applied to the body part of the user through the electrode patch in the (i+N+1)-th time interval.

### BRIEF DESCRIPTION OF THE DRAWINGS

FIG. 1 is a schematic diagram of an electronic device according to an embodiment of the disclosure.

FIG. 2 is a flowchart of a method for determining the intensity of a low-frequency current according to an embodiment of the disclosure.

FIG. 3 is a waveform diagram of electromyography values according to an embodiment of the disclosure.

FIG. 4 is a schematic diagram of determining a second current corresponding to an (i+N+1)-th time interval according to FIG. 3.

FIG. 5 is a flowchart of a mechanism of judging to stop applying a current according to an embodiment of the disclosure.

FIG. 6 is a schematic diagram of median frequency values according to FIG. 3.

### DETAILED DESCRIPTION OF DISCLOSED EMBODIMENTS

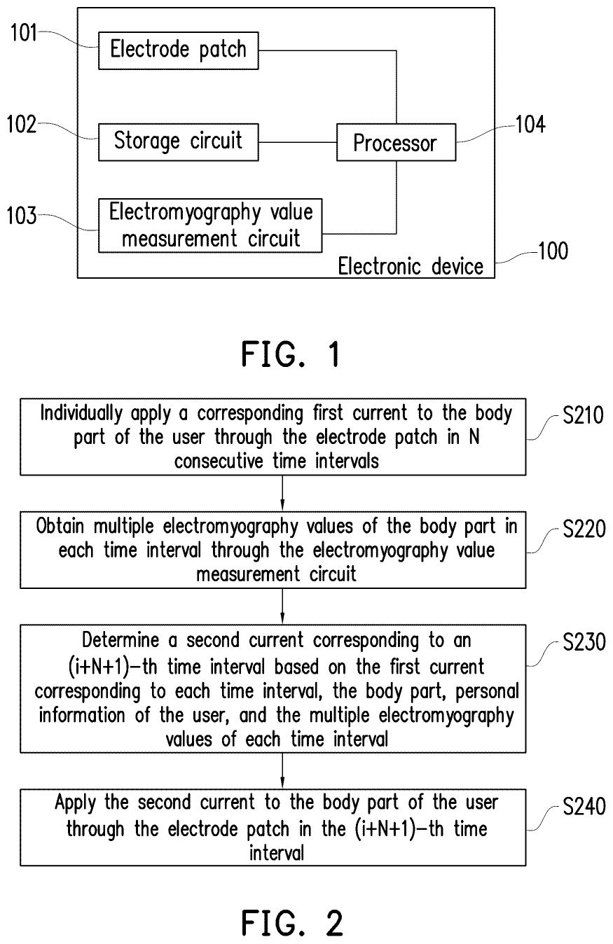

Please refer to FIG. 1, which is a schematic diagram of an electronic device according to an embodiment of the disclosure. In different embodiments, an electronic device 100 is, for example, a portable low-frequency therapy instrument or other similar devices/instruments, but not limited thereto.

As shown in FIG. 1, the electronic device 100 includes an electrode patch 101, a storage circuit 102, an electromyography value measurement circuit 103, and a processor 104. In the embodiment of the disclosure, the electrode patch 101 may be, for example, attached to a body part of a user who intends to undergo electrotherapy, and the processor 104 may be used to determine a current to be applied to the body part through the electrode patch 101, but not limited thereto. The electromyography value measurement circuit 103 may, for example, have a corresponding electrode patch (for example, the electrode patch 101 or other additional electrode patches), and the electrode patch may be attached to the body part to measure electromyography values of the body part, but not limited thereto.

The storage circuit 102 is, for example, any type of fixed or removable random access memory (RAM), read-only memory (ROM), flash memory, hard disk, other similar devices, or a combination of these devices that may be used to record multiple codes or modules.

The processor 104 is coupled to the storage circuit 102 and may be a general-purpose processor, a specific-purpose processor, a traditional processor, a digital signal processor, multiple microprocessors, one or more microprocessors combined with a digital signal processor core, controllers, microcontrollers, application specific integrated circuits (ASIC), field programmable gate array (FPGA), any other types of integrated circuits, state machines, advanced reduced instruction set computer (RISC) machine (ARM) processors, and similar products.

In the embodiment of the disclosure, the processor 104 may access modules and codes recorded in the storage circuit 102 to implement the method for determining the intensity of a low-frequency current proposed by the disclosure. The details thereof are as follows.

Please refer to FIG. 2, which is a flowchart of a method for determining the intensity of a low-frequency current according to an embodiment of the disclosure. The method of this embodiment may be executed by the electronic device 100 of FIG. 1. The details of each step in FIG. 2 will be described below in conjunction with the elements shown in FIG. 1.

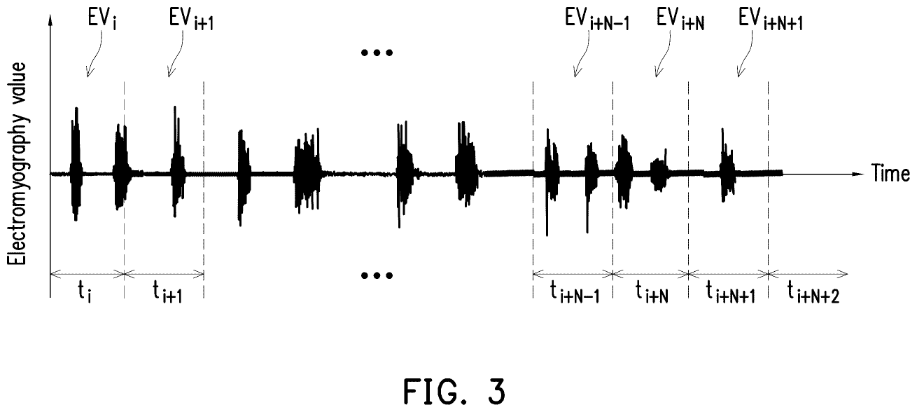

First, in Step S210, the processor 104 may individually apply a corresponding first current to the body part of the user through the electrode patch 101 in N consecutive time intervals. Next, in Step S220, the processor 104 may obtain multiple electromyography values of the body part in each time interval through the electromyography value measurement circuit 103. For ease of understanding the concept of the disclosure, the following will be further described with reference to FIG. 3.

Please refer to FIG. 3, which is a waveform diagram of electromyography values according to an embodiment of the disclosure. In FIG. 3, it is assumed that the processor 104 individually applies the corresponding first current to the body part of the user through the electrode patch 101 in N consecutive time intervals tᵢ to t_{i+N} (that is, an i-th time interval to an (i+N)-th time interval), and the processor 104 obtains the electromyography values of the body part in each time interval through the electromyography value measurement circuit 103.

In the embodiment of the disclosure, the electromyography values measured in the time interval tᵢ may be, for example, integratedly expressed as an electromyography value EVᵢ, and the electromyography values measured in the time interval tᵢ₊₁ may be, for example, integratedly expressed as an electromyography value EVᵢ₊₁. Accordingly, the meaning of the electromyography values EV_{i+N-1} to EV_{i+N+1} corresponding to the time intervals t_{i+N-1} to t_{i+N+1} may be deduced by analogy, which will not be repeated.

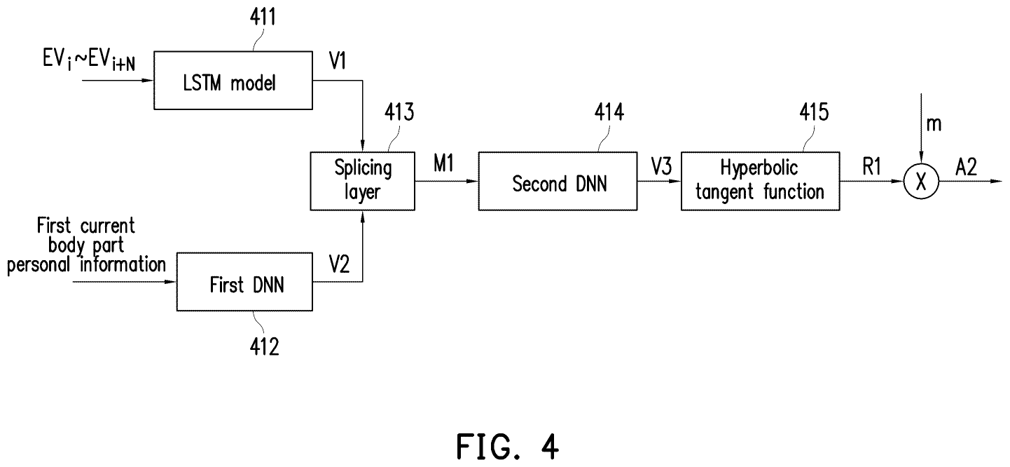

Then, in Step S230, the processor 104 may determine a second current corresponding to an (i+N+1)-th time interval (that is, the time interval t_{i+N+1}) based on the first current corresponding to each time interval tᵢ to t_{i+N}, the body part, personal information of the user, and the multiple electromyography values EVᵢ to EV_{i+N} of each time interval tᵢ to t_{i+N}. For ease of understanding, the details of Step S230 will be described below with reference to FIG. 4.

Please refer to FIG. 4, which is a schematic diagram of determining a second current corresponding to an (i+N+1)-th time interval according to FIG. 3. In an embodiment, the processor 104 may convert the multiple electromyography values EVᵢ to EV_{i+N} in each time interval tᵢ to t_{i+N} into a first vector V1. In FIG. 4, the processor 104 may, for example, first obtain a long short term memory (LSTM) model 411 including a 1-st hidden layer to an N-th hidden layer connected in series. After that, the processor 104 may respectively input the electromyography values EVᵢ to EV_{i+N} into the 1-st hidden layer to the N-th hidden layer. Correspondingly, the LSTM model 411 may generate the first vector V1 in response to the electromyography values EVᵢ to EV_{i+N}, so as to characterize the correlation between the electromyography values EVᵢ to EV_{i+N}, but not limited thereto.

In addition, the processor 104 may convert the first current corresponding to each time interval, the body part, and the personal information (for example, gender, age, etc.) of the user into a second vector V2, and splice the first vector V1 and the second vector V2 into a specific matrix M1.

In FIG. 4, the processor 104 may, for example, input the first current corresponding to each time interval, the body part, and the personal information of the user into a first deep neural network (DNN) 412. The first DNN 412 may generate the second vector V2 in response to the first current corresponding to each time interval, the body part, and the personal information of the user, but not limited thereto. After that, the processor 104 may splice the first vector V1 and the second vector V2 into the specific matrix M1 in a left-right or up-down splicing manner through a splicing layer 413, but not limited thereto.

Then, the processor 104 may convert the specific matrix M1 into a third vector V3, and convert the third vector V3 into a reference coefficient R1. In FIG. 4, the processor 104 may, for example, input the specific matrix M1 into a second DNN 414. The second DNN 414 may generate the third vector V3 in response to the specific matrix M1, but not limited thereto. In addition, the processor 104 may, for example, input the third vector V3 into a hyperbolic tangent function 415 to convert the third vector V3 into the reference coefficient R1 between −1 and 1, but not limited thereto.

After that, the processor 104 may multiply the reference coefficient R1 by a constant m to generate a second current A2 corresponding to the time interval t_{i+N+1}.

In Step S240, the processor 104 may apply the second current A2 to the body part of the user through the electrode patch 101 in the (i+N+1)-th time interval. In this way, the method of the disclosure may more appropriately determine the second current A2 corresponding to the time interval t_{i+N+1} after comprehensively considering the electromyography values in the past N time intervals tᵢ to t_{i+N}, the first current corresponding to each time interval tᵢ to t_{i+N}, the personal information of the user, and the body part attached to the electrode patch 101.

In addition, for the (i+N+2)-th time interval (that is, the time interval t_{i+N+2}), the processor 104 may still execute a mechanism similar to the above teaching to determine the third current corresponding to the time interval t_{i+N+2}.

Roughly speaking, the processor 104 may obtain the electromyography value EV_{i+N+1} of the body part in the time interval t_{i+N+1} through the electromyography value measurement circuit 103; determine the third current corresponding to the time interval t_{i+N+2} based on the second current A2, the body part, the personal information of the user, and the electromyography values EVᵢ₊₁ to EV_{i+N+1}; and apply the third current to the body part of the user through the electrode patch 101 in the time interval t_{i+N+2}. For the details of the above steps, please refer to the relevant descriptions in FIG. 2 to FIG. 4, which will not be repeated.

It can be seen from the above that the disclosure may determine the current used by the next time interval after comprehensively considering the electromyography values in the past N time intervals, the current corresponding to each time interval, the personal information of the user, and the body part attached to the electrode patch 101, thereby achieving the effect of intelligently adjusting the current intensity used during low-frequency therapy. In this way, poor therapeutic effect due to the user improperly selecting the current intensity may be prevented.

In some embodiments, in addition to the current intensity applied to the body part, the time length of the overall therapy is also a very important factor. The therapeutic effect is correspondingly reduced due to the therapy time being too long or too short. Therefore, the disclosure also proposes the following mechanism, which may be used to determine when to stop applying a current to the body part of the user, as detailed below.

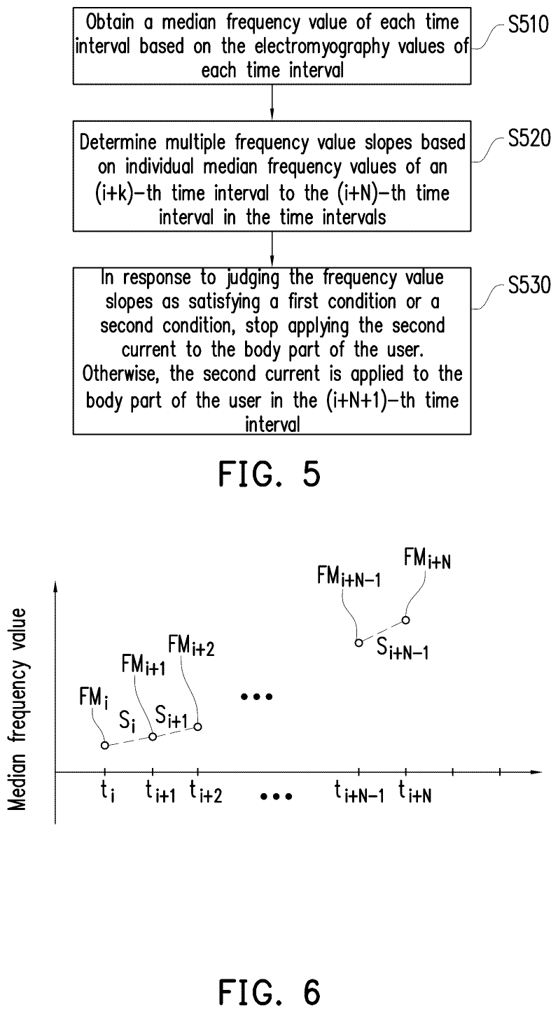

Please refer to FIG. 5, which is a flowchart of a mechanism of judging to stop applying a current according to an embodiment of the disclosure. The method of this embodiment may be executed by the electronic device 100 of FIG. 1. The details of each step of FIG. 5 will be described below in conjunction with the elements shown in FIG. 1. In addition, for ease of understanding the concept of the disclosure, the following will be described with reference to the scenarios of FIG. 3 and FIG. 6. FIG. 6 is a schematic diagram of median frequency values according to FIG. 3.

Generally speaking, before executing Step S240 of FIG. 2, the processor 104 may first execute each step in FIG. 5 to determine whether to continue applying the second current A2 in the time interval t_{i+N+1}.

First, in Step S510, the processor 104 may obtain a median frequency value of each time interval tᵢ to t_{i+N} based on the electromyography values EVᵢ to EV_{i+N} of each time interval tᵢ to t_{i+N}. Taking the time interval tᵢ as an example, the processor 104 may perform computations such as the Fourier transformation and normalization integration on the electromyography value EVᵢ to estimate a median frequency value FMᵢ corresponding to the time interval tᵢ. Taking the time interval tᵢ₊₁ as another example, the processor 104 may perform computations such as the Fourier transform and normalization integration on the electromyography value EVᵢ₊₁ to estimate a median frequency value FMᵢ₊₁ corresponding to the time interval tᵢ₊₁. For other time intervals (for example, the time intervals t_{i+N-1} and t_{i+N}), the processor 104 may estimate the corresponding median frequency value (for example, median frequency values FM_{i+N-1} and FM_{i+N}) based on a similar mechanism, but not limited thereto.

After that, in Step S520, the processor 104 may determine multiple frequency value slopes based on individual median frequency values of an (i+k)-th time interval (that is, the time interval tᵢ₊ₖ) to the (i+N)-th time interval in the time intervals tᵢ to t_{i+N}, where k≤N−1. In different embodiments, the designer may determine the value of k according to requirements. In the following descriptions, k is exemplified as 0, but not limited thereto.

When k is 0, the processor 104 may determine multiple frequency value slopes based on individual median frequency values of the time intervals tᵢ to t_{i+N}. In FIG. 6, the processor 104 may estimate the slope between the median frequency values FMᵢ and FMᵢ₊₁ as a frequency value slope Sᵢ. Similarly, the processor 104 may estimate the slope between the median frequency values FMᵢ₊₁ and FMᵢ₊₂ as a frequency value slope Sᵢ₊₁. Based on this principle, the processor 104 may determine frequency value slopes Sᵢ to S_{i+N-1} based on the individual median frequency values of the time intervals tᵢ to t_{i+N}.

After that, in Step S530, in response to judging the frequency value slopes Sᵢ to S_{i+N-1} as satisfying a first condition or a second condition, the processor 104 may stop applying the second current A2 to the body part of the user. Otherwise, the second current A2 may be applied to the body part of the user in the time interval t_{i+N+1} through the electrode patch 101.

In an embodiment, in response to judging n consecutive frequency value slopes Sᵢ to S_{i+N-1} (n may be determined by the designer according to requirements) as all positive and individual absolute values thereof as all less than a first preset value (for example, 0.1), the processor 104 may judge that the frequency value slopes Sᵢ to S_{i+N-1} satisfy the first condition. Otherwise, the frequency value slopes Sᵢ to S_{i+N-1} are judged as not satisfying the first condition.

Specifically, the median frequency values FMᵢ to FM_{i+N} corresponding to the time intervals tᵢ to t_{i+N}. may be respectively used to characterize indicators of muscle states in each time interval tᵢ to t_{i+N}. A lower median frequency value represents that the muscles of the body part are in a fatigue or tight state, while a higher median frequency value represents that the muscles of the body part are in a non-fatigue or relaxed state.

When the processor 104 judges that the n consecutive frequency value slopes Sᵢ to S_{i+N-1} are all positive and the individual absolute values thereof are all less than the first preset value (for example, 0.1), it represents that the body part of the user is gradually relaxed, so it can be known that a certain degree of therapeutic effect has been obtained. Therefore, the processor 104 may correspondingly stop applying the second current A2 to stop continuing to treat the body part of the user. On the other hand, when the processor 104 judges that the frequency value slopes Sᵢ to S_{i+N-1} do not satisfy the first condition, it represents that the body part of the user may need further therapy, so the processor 104 may correspondingly execute Step S240, but not limited thereto.

In an embodiment, in response to judging the n consecutive frequency value slopes Sᵢ to S_{i+N-1} as all negative or the individual absolute values thereof as all greater than a second preset value (for example, 0.6), the processor 104 may judge that the frequency value slopes Sᵢ to S_{i+N-1} satisfy the second condition. Otherwise, the frequency value slopes Sᵢ to S_{i+N-1} may be judged as not satisfying the second condition.

Specifically, when the processor 104 judges that the n consecutive frequency value slopes Sᵢ to S_{i+N-1} are all negative or the individual absolute values thereof are all greater than the second preset value, it represents that the body part of the user has not obtained the therapeutic effect, but shows worsening conditions such as more tightness and fatigue. Therefore, the processor 104 may correspondingly stop applying the second current A2 to stop continuing to treat the body part of the user. On the other hand, when the processor 104 judges that the frequency value slopes Sᵢ to S_{i+N-1} do not satisfy the second condition, it represents that the body part of the user may still undergo further therapy, so the processor 104 may correspondingly execute Step S240, but not limited thereto.

It can be seen from the above that the disclosure may appropriately determine when to stop applying a current to the user, thereby preventing the user from being unable to obtain a better therapeutic effect due to the therapy time being too long or too short.

In summary, the disclosure may determine the current used by the next time interval after comprehensively considering the electromyography values in the past N time intervals, the current corresponding to each time interval, the personal information of the user, and the body part, thereby achieving the effect of intelligently adjusting the current intensity used during low-frequency therapy. In this way, poor therapeutic effect due to the user improperly selecting the current intensity may be prevented.

In addition, the disclosure may also determine whether the body part of the user has obtained a certain degree of therapeutic effect or has worsened according to the changing condition of the median frequency values corresponding to each time interval. When it is judged that the body part of the user has obtained good therapy or has worsened, the disclosure may stop the therapy in time to prevent the user from being unable to obtain a better therapeutic effect due to the therapy time being too long or too short.

Although the disclosure has been disclosed in the above embodiments, the embodiments are not intended to limit the disclosure. Persons skilled in the art may make some changes and modifications without departing from the spirit and scope of the disclosure. The protection scope of the disclosure shall be defined by the appended claims.

What is claimed is:

1. A method for determining an intensity of a low-frequency current, applicable to an electronic device, comprising:
individually applying a corresponding first current to a body part of a user through an electrode patch in N consecutive time intervals, wherein the time intervals comprise an i-th time interval to an (i+N)-th time interval, where i and N are positive integers;
obtaining a plurality of electromyography values of the body part in each of the time intervals through an electromyography value measurement circuit;
determining a second current corresponding to an (i+N+1)-th time interval based on the first current corresponding to each of the time intervals, the body part, at least one personal information of the user, and the electromyography values of each of the time intervals, comprising:
converting the electromyography values in each of the time intervals into a first vector;
converting the first current corresponding to each of the time intervals, the body part, and the at least one personal information of the user into a second vector, and splicing the first vector and the second vector into a specific matrix;
converting the specific matrix into a third vector, and converting the third vector into a reference coefficient; and
multiplying the reference coefficient by a constant to generate the second current corresponding to the (i+N+1)-th time interval; and
applying the second current to the body part of the user in the (i+N+1)-th time interval through the electrode patch.

2. The method according to claim 1, wherein the step of converting the electromyography values in each of the time intervals into the first vector comprises:
obtaining a long short term memory model, wherein the long short term memory model comprises a 1-st hidden layer to an N-th hidden layer connected in series;
respectively inputting the individual electromyography values of the i-th time interval to the (i+N)-th time interval into the 1-st hidden layer to the N-th hidden layer, wherein the long short term memory model generates the first vector in response to the individual electromyography values of the i-th time interval to the (i+N)-th time interval.

3. The method according to claim 1, wherein the step of converting the first current corresponding to each of the time intervals, the body part, and the at least one personal information of the user into the second vector comprises:
inputting the first current corresponding to each of the time intervals, the body part, and the at least one personal information of the user into a first deep neural network, wherein the first deep neural network generates the second vector in response to the first current corresponding to each of the time intervals, the body part, and the at least one personal information of the user.

4. The method according to claim 1, wherein the step of converting the specific matrix into the third vector comprises:
inputting the specific matrix into a second deep neural network, wherein the second deep neural network generates the third vector in response to the specific matrix.

5. The method according to claim 1, wherein the step of converting the third vector into the reference coefficient comprises:
converting the third vector into the reference coefficient based on a hyperbolic tangent function, wherein the reference coefficient is between −1 and 1.

6. The method according to claim 1, further comprising:
obtaining a plurality of electromyography values of the body part in the (i+N+1)-th time interval through the electromyography value measurement circuit;
determining a third current corresponding to an (i+N+2)-th time interval based on the second current, the body part, the at least one personal information of the user, and the individual electromyography values of the (i+1)-th time interval to the (i+N+1)-th time interval; and
applying the third current to the body part of the user through the electrode patch in the (i+N+2)-th time interval.

7. The method according to claim 1, wherein before the step of applying the second current to the body part of the user through the electrode patch in the (i+N+1)-th time interval, the method further comprises:
obtaining a median frequency value of each of the time intervals based on the electromyography values of each of the time intervals;
determining a plurality of frequency value slopes based on the individual median frequency values of an (i+k)-th time interval to the (i+N)-th time interval in the time intervals, where k≤N−1; and
stopping to apply the second current to the body part of the user in response to judging the frequency value slopes as satisfying a first condition or a second condition, otherwise applying the second current to the body part of the user through the electrode patch in the (i+N+1)-th time interval.

8. The method according to claim 7, wherein in response to judging n consecutive frequency value slopes as all positive and individual absolute values thereof as all less than a first preset value, the frequency value slopes are judged as satisfying the first condition, otherwise the frequency value slopes are judged as not satisfying the first condition.

9. The method according to claim 7, wherein in response to judging n consecutive frequency value slopes as all negative or individual absolute values thereof as greater than a second preset value, the frequency value slopes are judged as satisfying the second condition, otherwise the frequency value slopes are judged as not satisfying the second condition.
